(19) United States
(12) Patent Application Publication
(10) Pub. No.: US 2007/0254328 A1
(43) Pub. Date: Nov. 1, 2007

(54) Glyoxylate assays and their use of inden tifying natural amidated compounds

(75) Inventors: Sarah E. Carpenter, Caldwell, NJ (US); Dave J. Merkler, Valrico, FL (US); Duncan A. Miller, Morris Township, NJ (US); Nozer M. Mehta, Randolph, NJ (US); Angelo P. Consalvo, Monroe, NY (US)

Correspondence Address: OSTROLENK FABER GERB & SOFFEN, 1180 AVENUE OF THE AMERICAS, NEW YORK, NY, 100368403, US

(73) Assignee: Unigene Laboratories Inc.

(21) Appl. No.: 11/654,211

(22) Filed: Jan. 17, 2007

Related U.S. Application Data
(60) Provisional application No. 60/761,681, filed on Jan. 23, 2006.

(51) Int. Cl. C12Q 1/26

(52) U.S. Cl. 435/25

(57) ABSTRACT

Methods for detecting and assaying for glyoxylate, include enzyme-based assays and/or assays for hydrogen peroxide following liberation of hydrogen peroxide from glyoxylate, are disclosed. In some embodiments, the invention is directed to methods for assaying for glyoxylate produced by the reaction of peptidylglycine alpha-amidating monooxygenase (PAM). The subject invention also concerns methods for assaying for the enzyme peptidylglycine alpha-amidating monooxygenase and/or its substrates. The detection of glyoxylate is indicative of the presence of PAM and/or its substrates. The subject invention also concerns methods for screening for peptide hormones, amidated fatty acids, any N-acyl-glycine or N-aryl-glycine conjugated molecule, and generally compounds having a glycine reside in free acid form and attached to a carbonyl group.

This invention was made with government support under the National Institute of Health SBIR grant number 1-R43-DK063812-01. The government has certain rights in the invention.

### BACKGROUND OF THE INVENTION

C-Terminal amidation of glycine extended prohormones is a post-translational modification necessary for the activity of many peptide hormones. Amidated peptide hormones are an important class of hormones found in mammals, insects, and cnidarians. The discovery of novel amidated hormones has been severely hindered by the lack of a good assay specific to this class of bioactive hormones. The formation of all amidated peptide hormones is dependant upon the activity of Peptidylglycine alpha-Amidating Monooxygenase (PAM). Glycine extended precursors reacted with PAM result in the formation of an alpha-amidated peptide and glyoxylate. The general reaction is shown below in Scheme 1:

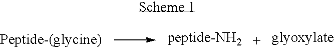

Glyoxylate (HCO—COO⁻), a product of the reaction shown in Scheme 1, is a metabolite synthesized and catabolized by both vertebrates and invertebrates (Gragera, R. R. et al. (2000) “Localization of Glyoxylate Dehydrogenase and Glyoxylate Complex Molecules in the Rat Prefrontal Cortex: enzymohistochemical and Immnuocytochemical Study” J. Neurosci. Res. 59:561-567). Calcium oxalate is the major constituent of kidney stones (Asplin, 2002) and approximately 50-60% of urinary oxalate (⁻OOC—COO⁻) is derived from the enzymatic oxidation of glyoxylate (HCO—COO⁻) (Williams, H.E. (1989) “Oxalate Synthesis, Transport and the Hyperoxaluric Syndromes: J. Urol. 141:742-749).

As a consequence of the metabolic importance and role of glyoxylate in kidney stone formation, a number of assays have been developed for glyoxylate. Existing assays for the determination of glyoxylate include colorimetric methods (Albrecht, A. M. et al. (1962) “Determination of Aliphatic Aldehydes by Spectrophotometry” Anal. Chem. 34:398-400; Soda, K. et al. (1973) “Spectrophotometric Determination of Glyoxylic Acid with o-aminobenzaldehyde and Glycine, and its Application to Enzyme Assay” Agr. Biol. Chem. 37:1393-1400; Bongers, J. et al. (1992) “Semisynthesis of Human Growth Hormone-Releasing Factors by α-Amidating Enzyme Catalyzed Oxidation of Glycine-Extended Precursors” Peptide Res. 5:183-189; Kramer, D. N. et al. (1959) “Quantitative Determination of Glyoxylic Acid” Anal. Chem. 31:250-252; Vogels, G. D. et al. (1970) “Differential Analyses of Glyoxylate Derivatives” Anal. Biochem 33:143-157, fluorometric methods (Spikner, J. E. et al. (1962) “Fluorometric Microdetermination of Alpha-Keto Acids” Anal. Chem. 34:1468-1471; Zarembski, P. M. et al. (1965) “The Fluorometric Microdetermination of Glyoxylic Acid in Blood, Urine and Bacterial Extracts” Biochem. J. 96:218-223, the iodometric or potentiometric titration of the bisulfite adduct (McFadden, B. A. et al. (1960) “The Determination of Glyoxylic Acid in Biological Systems” Anal. Biochem. 1:240-248, and the use of capillary electrophoresis with direct UV detection (Nishijima, S. et al. (2001) “Glyoxylate Determination in Rat Urine by Capillary Electrophoresis” Int. J. Urol. 8:S63-S67; Garcia, A. et al. (2001) “Measurement of Nephrolithiasis Urinary Markers by Capillary Electrophoresis” J. Chromatogr. B 755:287-295.

Generally, these assays are insensitive and/or nonspecific. Increased sensitivity has been provided by the separation and quantification of the colored or fluorescent glyoxylate derivative by HPLC (Bongers, J. et al. (1992) “Semisynthesis of Human Growth Hormone-Releasing Factors by α-Amidating Enzyme Catalyzed Oxidation of Glycine-Extended Precursors” Peptide Res. 5:183-189; Funai, T. et al. (1986) “High-Performance Liquid Chromatographic Determination of Glyoxylate in Rat Liver” J. Biochem 99:579-589; Mentasi, E. et al. (1987) “High-Performance Liquid Chromatographic Determination of Glyoxylic Acid and other Carboxyl Compounds in Urine” J. Chromatogr. B. 417:253-260, 1987; Petrarulo, M. et al. (1988) “High-Performance Liquid Chromatographic Determination of Glyoxylic Acid in Urine” J. Chromatogr. 432:37-46; Lange, M. et al. (1994) “Fast Method for the Simultaneous Determination of 2-Oxo Acids in Biological Fluids by High-Performance Liquid Chromatography” J. Chromatogr. B 662:97-10). However, specificity remains less than ideal with these techniques. Thus, there remains a need in the art for a rapid, specific, sensitive assay for glyoxylate.

### BRIEF SUMMARY OF THE INVENTION

The subject invention concerns methods for detecting and assaying for glyoxylate, following conversion of glyoxylate to, inter alia, hydrogen peroxide. Assays utilizing several different enzymes for assaying for glyoxylate are provided herein. Enzymatic liberation of hydrogen peroxide from glyoxylate is contemplated. Exemplified herein are assays wherein detection is accomplished using spectrophotometry, fluorescence, or luminescence.

The subject invention also concerns methods for assaying for the enzyme peptidylglycine alpha-amidating monooxygenase (PAM). The detection of glyoxylate using the present invention is frequently indicative of the presence of PAM and its substrates. PAM is known to oxidatively cleave glycine-extended peptide and fatty acid substrate prohormones to their corresponding amidated product and glyoxylate in an equimolar ratio. Glycine-extended prohormones are frequently relatively inactive prior to PAM dependent amidation. Moreover, PAM regulates hormonal activity by amidating glycine extended substrates. Therefore assaying for PAM activity by quantifying glyoxylate allows one to not only test PAM activity but to also assay a wide variety of glycine extended substrate derivatives. PAM activity can also be provided by PAM's two catalytic domains, peptidylglycine alpha-hydroxylating monooxygenase (PHM) and peptidyl alpha-hydroxyglycine alpha-amidating lyase (PAL) or by a combination of PAM and a Lewis base.

The subject invention also concerns methods for screening for peptide hormones and any N-acyl-glycine or N-aryl-glycine conjugated molecule. The detection of glyoxylate using the present invention suggests the presence of PAM. The presence of PAM likewise suggests that an alpha-amidated peptide is being produced. The subject invention provides a means for the discovery of novel hormones that regulate mammalian function.

In one embodiment, the invention provides a method for detecting the presence of glyoxylate in a test sample comprising the steps of contacting said test sample with one or more agents that convert glyoxylate, directly or indirectly, to hydrogen peroxide as one reaction product; and assaying for the presence of hydrogen peroxide.

In another embodiment, the invention provides a method for detecting the presence of compounds having a glycine residue, in free acid form and attached to a carbonyl group, said method comprising the steps of contacting a sample suspected of containing said glycine compounds with either (i) peptidylglycine alpha-amidating monooxygenase (PAM), (ii) a combination of peptidylglycine alpha-hydroxylating monooxygenase (PHM) and peptidyl alpha-hydroxyglycine alpha-amidating lyase (PAL) or (iii) a combination of peptidylglycine alpha-hydroxylating monooxygenase and a Lewis base; to form an amidated compound and glyoxylate; and assaying for the presence of glyoxylate.

In another embodiment, the invention provides, a method of identifying amidated hormones or amidated fatty acids comprising the steps of (A) incubating cells suspected of producing said hormones or fatty acids; (B) fractionating compounds produced by said cells into a series of fractions for further analysis; (C) contacting a fraction from step (B) with either (i) peptidylglycine alpha-amidating monooxygenase, (ii) a combination of peptidylglycine alpha-hydroxylating monooxygenase and peptidyl alpha-hydroxyglycine alpha-amidating lyase or (iii) a combination of peptidylglycine alpha-hydroxylating monooxygenase and a Lewis base; to form an amidated compound and glyoxylate when said fraction includes a compound having a glycine residue, in free acid form and attached to a carbonyl group; and (D) assaying the product of step (C) for the presence of glyoxylate.

### BRIEF DESCRIPTION OF THE DRAWINGS

FIG. 1 shows spectrophotometric malate synthase/malate dehydrogenase (MS/MD) enzyme-linked colorimetric assay for glyoxylate. Glyoxylate is measured by the malate synthase/malate dehydrogenase-dependent formation of an intensely colored formazan (1=malate synthase, 2=malate dehydrogenase, and PMS=phenazine methosulfate). PMS serves to shuttle electrons from NADH to the tetrazolium (MTS).

FIGS. 2A and 2B show glyoxylate-dependent oxidation of MTS. The increase in absorbance obtained using glyoxylate (FIG. 2A) and that obtained by the base-catalyzed dealkylation of α-hydroxyhippurate to benzamide and glyoxylate (FIG. 2B). The data points are the average of 3-10 determinations and the error bars represent the standard deviation of the measurements.

FIG. 3 shows lactate dehydrogenase-glycolate oxidase, glycolate oxidase reaction (LD-GO, GO). Glyoxylate is measured by the enzyme dependant oxidative coupling of two compounds which produce an indamine dye measurable at 590nm. The PAM substrate dns-YV-Gly was collected by Preparative HPLC, reacted with PAM, and the product DNS-YV-NH₂ was quantified by HPLC. Concentration of glyoxylate was determined from the 1:1 molar ratio of dns-YV-NH₂:glyoxylate. Standard Curve analysis of PAM produced glyoxylate is shown in

FIG. 4 to match the literature cited Indamine extinction coefficient. Slope=0.05000 FIG. 4 shows analysis of glyoxylate/glycine-extended peptide by the LD-GO, GO assay. This graph demonstrates that the dansylated tripeptide Tyr-Val-Gly can be quantitatively measured by production of hydrogen peroxide according to the literature value of 0.05300M⁻cm⁻.

FIG. 5 shows the glyoxylate reductase spectrophotometric assay for glyoxylate. The enzyme activity of glyoxylate reductase consumes a stoichiometric quantity of NADPH to glyoxylate, the loss of NADPH can be measured at 340 nm. The PAM substrate dns-TYGly was collected by Preparative HPLC, reacted with PAM, and the product DNS-TVNH₂ was quantified by HPLC. Concentration of glyoxylate was determined from the 1:1 molar ratio of dns-TYNH₂: glyoxylate. Standard Curve analysis of PAM produced glyoxylate is shown in FIG. 6 to match the literature cited NADPH extinction coefficient. Slope=0.0063.

FIG. 6 shows stoichiometric detection of glyoxylate and an α-amidated peptide. This graph demonstrates the ability of this glyoxylate assay to be applicable to the PAM assay system for the identification of α-amidated hormones. The literature value of NADPH consumption is 0.0062M⁻cm⁻.

FIGS. 7A and 7B show fluorescent enzymatic assays for glyoxylate. Both enzymatic assays utilize the oxidation of Amplex Red as the fluorescent analyte for detection of glyoxylate. The oxidation of Amplex Red is dependant upon the production of hydrogen peroxide. The reaction in FIG. 7A uses glycolate oxidase; The reaction in FIG. 7B uses glyoxal oxidase. The PAM substrate dns-YV-Gly was collected by Preparative HPLC, reacted with PAM, and the product dns-YV-NH₂ was quantified by HPLC. Concentration of glyoxylate was determined from the 1:1 molar ratio of dns-YV-NH₂: glyoxylate. Standard Curve analysis of PAM produced glyoxylate is shown in FIGS. 8A and 8B for each enzyme assay, and matches the standard curve for H₂O₂.

FIGS. 8A and 8B show stoichiometric detection of glyoxylate by fluorescence. Both assays show linear detection of both pure glyoxylate and PAM produced glyoxylate. The PAM substrate dansyl-Tyr-Val-Gly was independently quantified and used to quantitative PAM produced glyoxylate which was detected in this assay. Results from the reaction of FIG. 7A are shown in FIG. 8A and results from the reaction of FIG. 7B are shown in FIG. 8B.

FIG. 9 shows description of luminescent assay for glyoxylate. Both Glyoxal Oxidase and Glycolate Oxidase can be used for the luminescent detection assay. For detection of PAM produced glyoxylate the first two sets are necessary, and can be eliminated for detection of glyoxylate alone. The PAM substrate dns-YV-Gly was collected by Preparative HPLC, reacted with PAM, and the product dns-YV-NH₂ was quantified by HPLC. Concentration of glyoxylate was determined from the 1:1 molar ratio of dns-YV-NH₂: glyoxylate. Standard Curve analysis of PAM produced glyoxylate is shown in FIG. 11, and matches the standard curve for H₂O₂/glyoxylate.

FIG. 10 shows reaction mechanism for the production of light from luminol. Oxidation to the excited state of luminol is proportional to the quantity of hydrogen peroxide.

FIG. 11 shows glyoxylate oxidase luminescent detection of glyoxylate. This data demonstrates the ability of the luminescent assay to detect the presence of a glycine-extended peptide via glyoxylate.

FIG. 12 shows flow chart of independent analysis of accumulated peptides by both luminescence, and MALDI-TOF.

FIGS. 13A and 13B show HPLC fractions collected and assayed for glyoxylate.

FIG. 13A shows detection of spiked cell culture spiked mJP-Gly (2.5 nmoles) by luminescent analysis of fractions for glyoxylate. FIG. 13B shows detection of mJP-Gly accumulated in cell culture by the presence of a PAM inhibitor, by the luminescent assay for PAM. Identification of mJP-Gly via glyoxylate in the same fraction is evidence that the glyoxylate observed is derived from the PAM dependant conversion of mJP-Gly to α-amidated-mJP and glyoxylate.

FIGS. 14A-14D show identification of glyoxylate positive fraction for the presence of mJP-Gly. Fractions 31 from both a spiked sample (FIGS. 14C and 14D) and non-spiked sample (FIGS. 14A and 14B) were assayed for the presence of mJP by MALDI-TOF Mass Spectrometry. Identification of the glycine-extended form demonstrates that indeed PAM was inhibited in cell culture, and the glyoxylate is coincident with the peptides analyzed by Mass Spectrometry.

FIG. 15 is a PAM/glycolate oxidase enzyme linked luminescent assay of RP-HPLC fractions 25 through 29 in Example 2 infra. Blanks or buffer washes are performed between fractions. Relative luminescence indicated the presence of rat CGRP-Gly in fraction 29.

FIG. 16 is a MALDI-TOF Mass Spectrometry analysis of fraction 29 in example 2 infra from conditioned medium purified by chromatography on a Hypersil BDS C18 column.

### DETAILED DISCLOSURE OF THE INVENTION

The subject invention concerns enzyme-based methods for detecting and assaying for glyoxylate. Glyoxylate is a molecule of interest to the scientific community as its in vivo production is signature of many health issues. Likewise, glyoxylate is involved in several plant biochemical pathways namely the “glyoxylate cycle”, and therefore analyzing glyoxylate concentration with this new technology will be of importance in the fields of plant biochemistry, botany, and horticulture.

In one embodiment, methods of the invention can be used for assaying for glyoxylate produced by the reaction of peptidylglycine alpha-amidating monooxygenase (or PHM+PAL, or PHM+Lewis base). The subject invention also concerns methods for assaying for the enzyme peptidylglycine alpha-amidating monooxygenase (or PHM+PAL, or PHM+Lewis base). The detection of glyoxylate using the present invention is indicative of the presence of PAM (or PHM+PAL, or PHM+Lewis base. In order to confirm activity by the foregoing enzyme(s) of interest, a fraction testing positive for glyoxylate following incubation with PAM (or PHM+PAL, or PHM+Lewis base) can be retested without first incubating with PAM (or PHM +PAL, or PHM+Lewis base). A large difference in glyoxylate formed with—versus without—PAM (or PHM+PAL, or PHM+Lewis base) indicates that the glyoxylate is formed by action of the enzyme(s) of interest.

In preferred embodiments, compounds in any fraction that tests positive for glyoxylate in accordance with the method of the invention, are further analyzed for characteristics of amidated peptide hormone precursors, said characteristics being selected from the group consisting of (1) having a C-terminal glycine in its amino acid sequence and (2) having, in its corresponding nucleotide sequence, a signal region in reading frame with a peptide coding region that either terminates with C-terminal glycine or has a glycine followed by single or multiple basic amino acids that serve as post-translational cleavage sites immediately downstream.

PAM is the key enzyme in the regulation of over 50% of all known hormones, and has been widely studied by the scientific community based on its extremely important physiological role. PAM is known to oxidatively cleave glycine-extended peptides and fatty acid substrate prohormones to their corresponding amidated product and glyoxylate in an equimolar ratio. Glycine-extended prohormones are often relatively inactive prior to PAM dependent amidation. Moreover, PAM regulates hormonal activity by amidating glycine extended substrates. Therefore, assaying for PAM activity by quantifying glyoxylate allows one to not only test for PAM activity but to also assay for a wide variety of glycine extended substrate derivatives.

The subject invention also concerns methods for screening for peptide hormones and any N-acyl-glycine or N-aryl-glycine conjugated molecule. The detection of glyoxylate using the present invention is indicative of the presence of PAM. The presence of PAM is likewise indicative that an α-amidated peptide is also being produced. Defining tissues that have high levels of PAM activity provides researchers with a place to search for novel peptide hormone substrates. Prior techniques, for the discovery of novel peptide hormones, are not believed to provide the combination of efficiency and sensitivity provided by the present invention. The subject invention provides a means for the discovery of novel hormones that regulate mammalian function. Thus, the present invention provides a series of assays specific for the discovery of numerous unidentified amidated hormones. The assays exploit this very unique biosynthetic pathway for the formation of the amidated peptides.

As exemplified herein, a series of enzyme dependent assays for glyoxylate with detection by, for example, spectrophotometry, fluorescence, and luminescence, have been developed. The assays of the present invention can use any suitable detection means and are not limited to those means specifically exemplified herein. Several areas can benefit from this technology ranging from the medical fields to research science. Three different spectrophotometric assays are exemplified herein, each of which utilizes different enzyme detection systems.

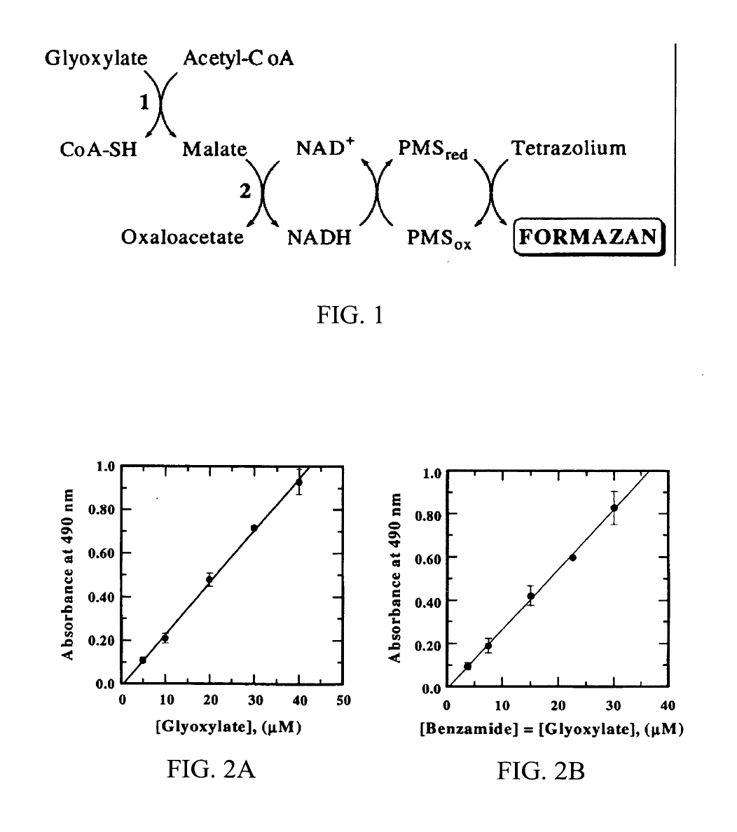

One glyoxylate assay that may be used to search for PAM activity or substrates in accordance with the present invention utilizes malate synthase/malate dehydrogenase in which enzymatically oxidized glyoxylate and acetyl-CoA produce oxaloacetate with the concomitant reduction of NAD⁺ to NADH. A sample to be assayed for glyoxylate is contacted with Acetyl-CoA and malate synthase and malate dehydrogenase. The presence of glyoxylate in the sample results in oxaloacetate and NADH production. In one embodiment, NADH produced is then detected using phenazine methosulfate (PMS) and a tetrazolium compound (MTS). NADH drives reduction of PMS which in turn drives the reduction of a tetrazolium compound (MTS) to produce an intensely colored reduced formazan with a VIS detection limit of approximately 5 nanomoles glyoxylate. A general reaction scheme for the assay is shown in FIG. 1 and results are shown in FIGS. 2A-2B. See also S. E. Carpenter and D. J. Merkler, “An Enzyme Coupled Assay for Glyoxylate”, Analytical Biochemistry, December 15, 2003, 323(2), pp. 242-246, the disclosure of which is incorporated herein by reference.

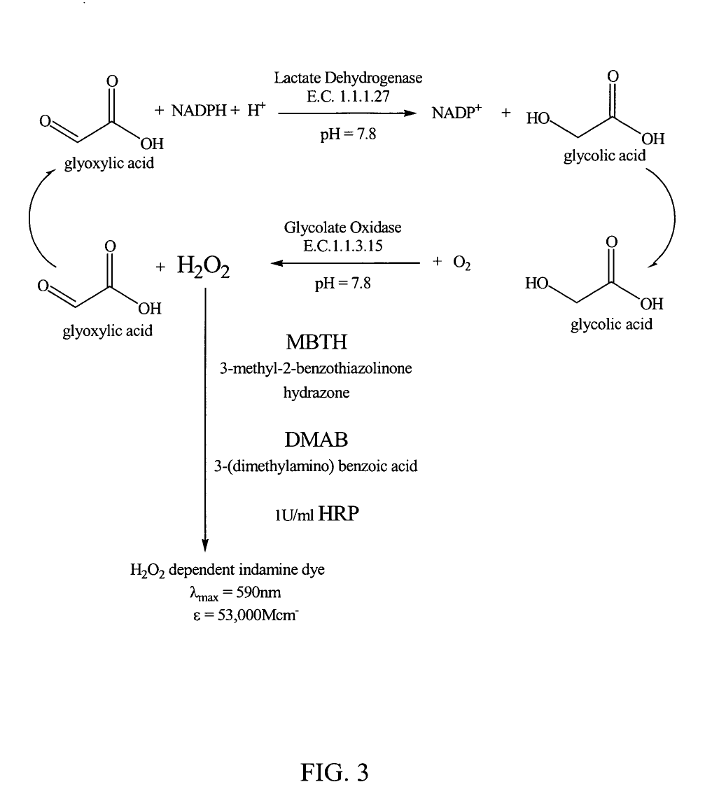

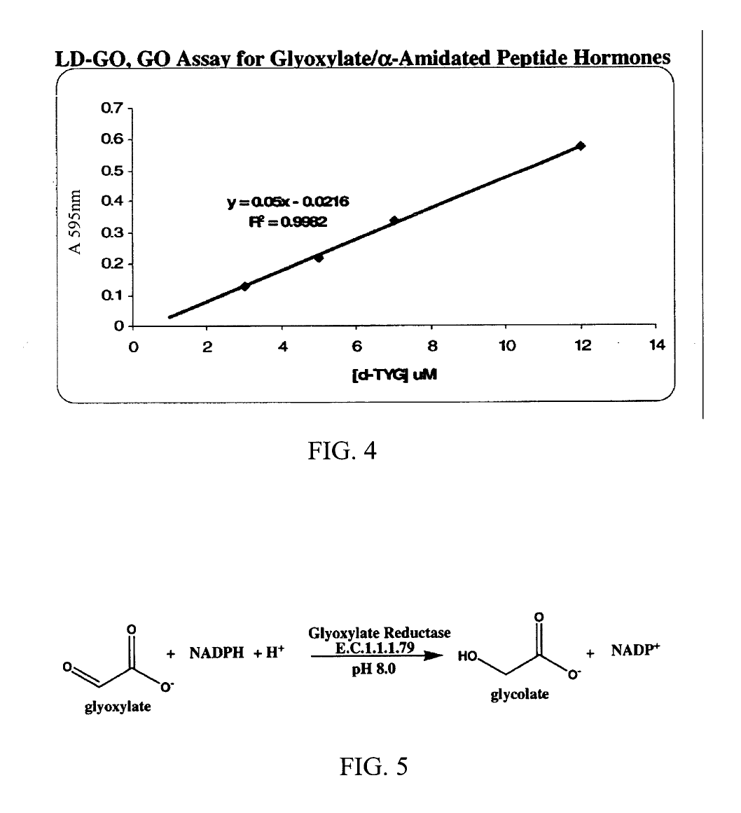

A further embodiment of the invention concerns an assay that utilizes lactate dehydrogenase/glycolate oxidase-glycolate oxidase (LD-GO,GO) which oxidizes glyoxylate to glycolate with a stoichiometric production of hydrogen peroxide. A sample to be assayed for glyoxylate is contacted with lactate dehydrogenase and glycolate oxidase. The hydrogen peroxide produced from the enzymatic reaction can then be detected by any of a variety of techniques. In one embodiment with spectrophotometric detection, an MBTH/DMAB-indamine dye detection system can be used based on its low detection limit for a spectrophotometric assay of 300 nmoles. A general reaction scheme for the lactate dehydrogenase/glycolate oxidase-glycolate oxidase assay is shown in FIG. 3 and results are shown in FIG. 4.

In another embodiment, the present invention concerns an assay that utilizes glycolate oxidase or glyoxal oxidase to produce oxalate and a stoichiometric amount of H₂O₂ from glyoxylate. The H₂O₂ produced from the reaction can then be detected by any of a variety of techniques. In one embodiment, a fluorescent-based detection method that utilizes Amplex Red and horseradish peroxidase are used to detect H₂O₂, wherein the Amplex Red is oxidized to the fluorescent molecule Resorufin. Other methods for detection of H₂O₂ are known in the art and are contemplated within the scope of the present invention.

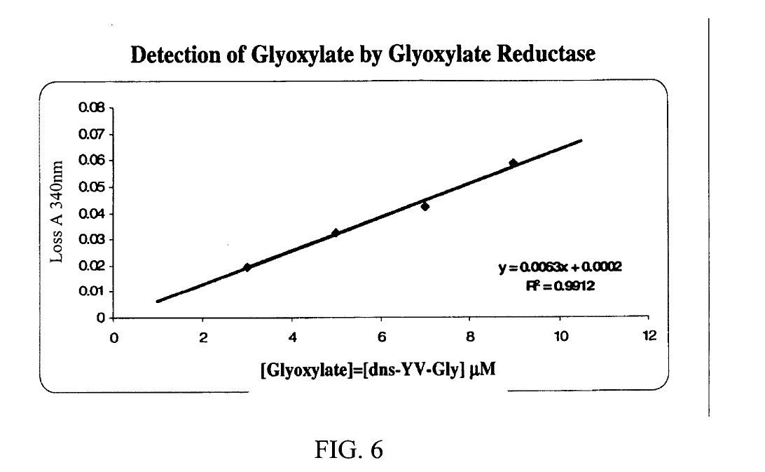

In a still further embodiment, the present invention concerns an assay that utilizes glyoxylate reductase in which glyoxylate is reduced to glycolate with the concomitant oxidation of NADPH to NADP⁺. Production of NADP⁺ results in a change in absorbance at 340 nm with a detection limit of 900 nmoles. A general reaction scheme for the assay is shown in FIG. 5 and results are shown in FIG. 6.

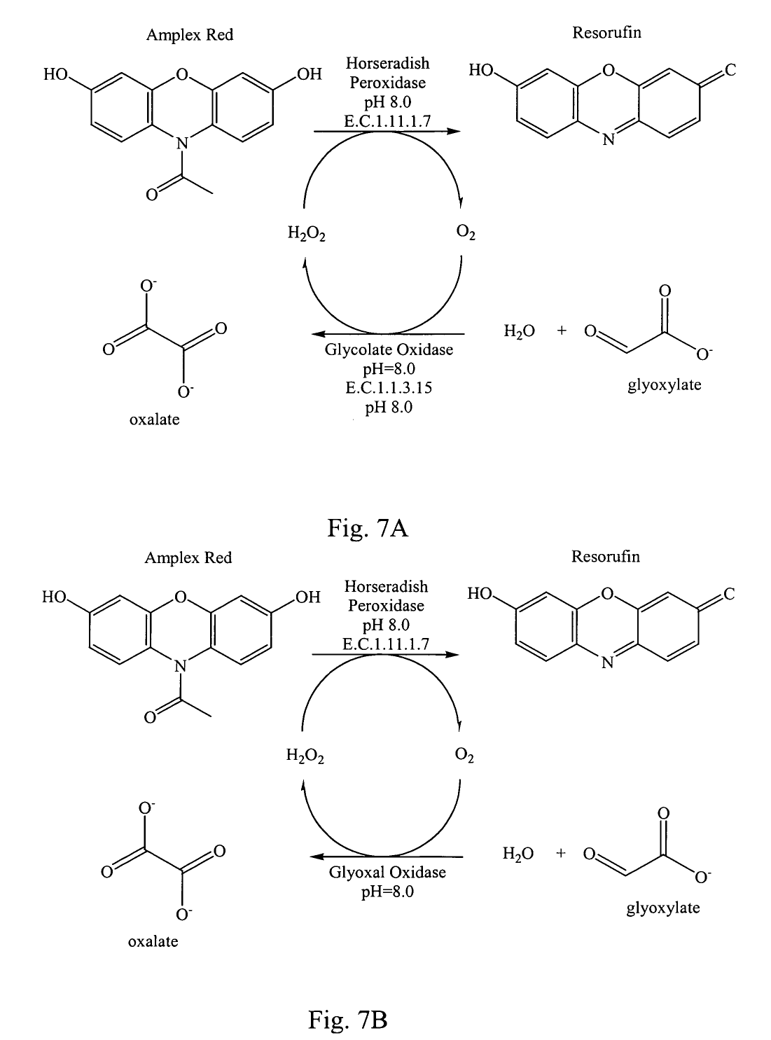

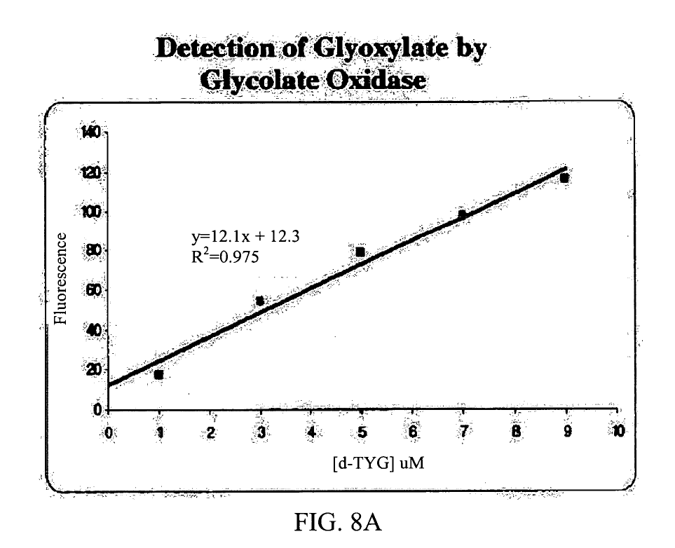

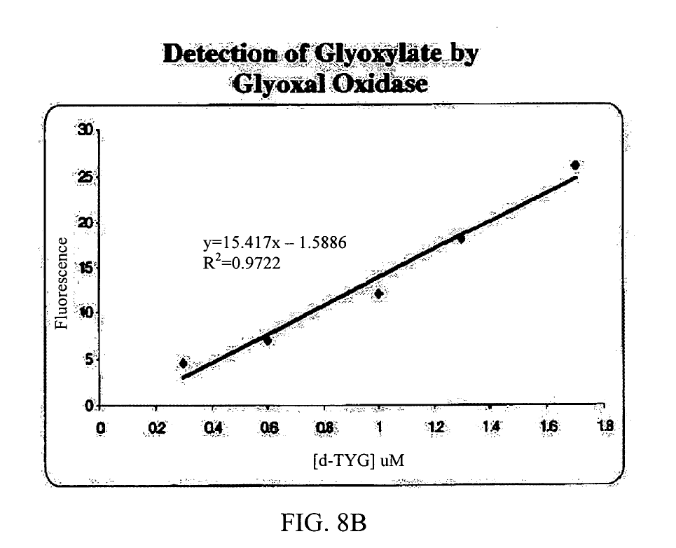

Fluorescence-based detection methods that can be used in the present invention are based on enzymne-dependant stoichiometric production of hydrogen peroxide to glyoxylate consumption. Hydrogen peroxide is detectable by a variety of techniques; one assay exemplified herein utilized Amplex Red, a non-fluorescent substrate for horse radish peroxidase which in the presence of hydrogen peroxide oxidizes to the highly fluorescent molecule Resorufin (see FIGS. 7A and 7B). Oxidation of Amplex Red is dependent upon the presence of hydrogen peroxide and this assay proved stoichiometric for the quantification of glyoxylate based on the chemistry of the chosen enzymatic reactions. To modify the assay for high through-put analysis, as well as sensitivity, the assay was modified to a microplate format with detection levels in the range of 10-30 pmole. Two enzymes were chosen for the fluorescent assay: glyoxal oxidase (the general reaction scheme is shown in FIG. 7B and results are shown in FIG. 8B) and glycolate oxidase (the general reaction scheme is shown in FIG. 7A and results are shown in FIG. 8A). Both enzymes produce stoichiometric quantities of hydrogen peroxide from glyoxylate. It is imperative that all FMN (flavin mononucleotide) be removed from the glycolate oxidase enzyme prior to glyoxylate analysis with Amplex Red. This molecule is oxidatively labile and will auto-oxidize in the presence of FMN. As all FMN must be removed from the glycolate oxidase enzyme, FAD (flavin adenine dinucleotide) is utilized as the flavin of choice for this reaction, as FAD also supports GO catalysis yet does not oxidize Amplex Red.

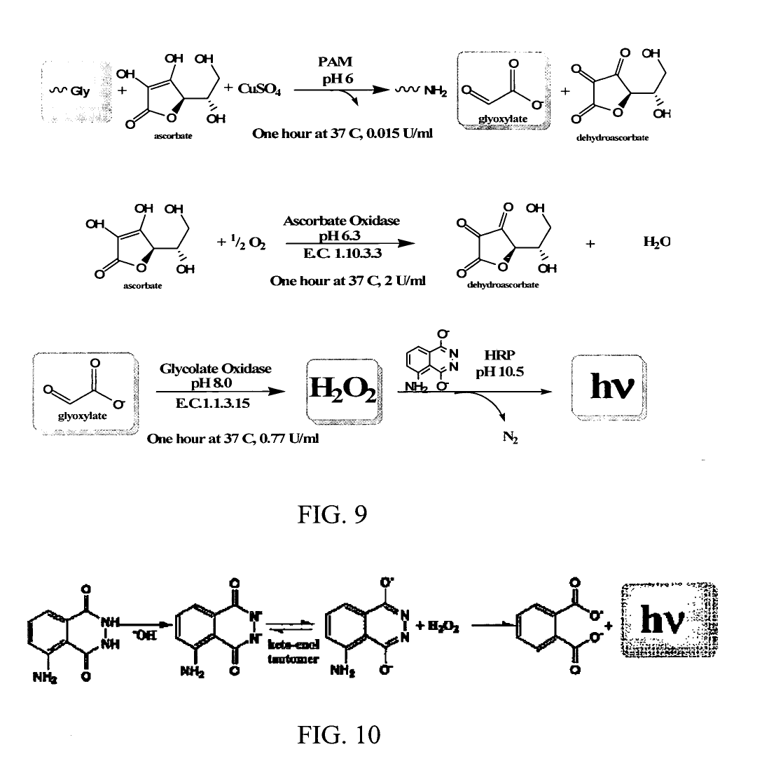

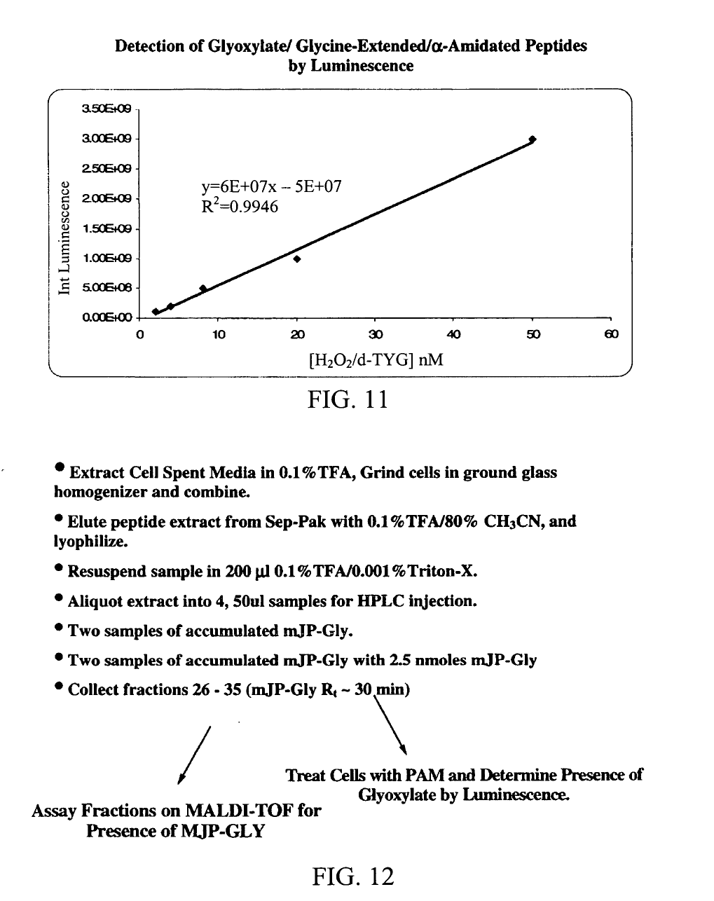

Luminescence based detection methods can also be used with the assays of the present invention and proved to be the most sensitive. An exemplified assay is based on the chemiluminescence of luminol. In the presence of an oxidative catalyst and a basic environment, luminol becomes excited into the triplet spin state in the presence of hydrogen peroxide. The relaxation of luminol back to the singlet state then releases a photon of light (see FIG. 10). The emission of light is concentration dependant, thereby affording a highly sensitive technique for analyzing hydrogen peroxide concentration in the femtomole region. Utilizing glycolate oxidase and/or glyoxal oxidase, a stoichiometric amount of hydrogen peroxide is produced (FIG. 9), thus providing the most sensitive of all techniques exemplified herein for the quantification of glyoxylate. Results using a luminescent assay of the invention are shown in FIG. 11.

Assays of the present invention can be used to screen for the presence of an amidated peptide in a sample. When cells grown in the presence of a PAM inhibitor, PAM substrates, such as glycine extended peptide will accumulate. Alternately, PAM can be inhibited by the introduction of RNAi into the cell to degrade PAM mRNA and prevent its translation into PAM protein. Cell extracts and/or spent media are prepared from the cells grown in the presence of a PAM inhibitor or grown in the presence of PAM specific RNAi. Chromatographic techniques, such as HPLC, can then be used to fractionate the cell extracts and/or spent media samples. The HPLC fractions can then be treated with PAM. The accumulated glycine extended peptides are acted on by PAM to produce the amidated peptide plus glyoxylate. The PAM treated fractions (from which ascorbate can be removed if necessary) can then be assayed for the presence of the glyoxylate (produced by the PAM reaction) using any assay of the present invention. Fractions which contain glyoxylate are positive for a glycine extended peptide. Glycine-extended peptides can be characterized by N-terminal sequence analysis and mass spectrometry to determine the identity of the amidated peptide.

Application of the assays of the invention to the quantification of PAM-produced glyoxylate required two alterations of the PAM assay, as well as one change to the common glycolate oxidase assay which has previously been mentioned. Without these specific alterations the assays would be rendered non-stoichiometric and produce anomalous data. First, to address the changes to the PAM assay, a new method for enzyme protection due to hydroxyl radical formation produced during catalysis was necessary as the use of catalase has previously been the method of choice. Catalase catalyzes the disproportionate reaction of H₂O₂ to H₂O and O₂, and removal of hydrogen peroxide is detrimental to the assay methods. Horseradish peroxidase was found to both protect the PAM enzyme, and not interfere with hydrogen peroxide detection. Second, a reducing agent is necessary for the PAM catalysis. Ascorbate is frequently used in the art for this purpose. However, in the present invention, ascorbate can both severely inhibit the spectrophotometric, fluorescent, and luminescent assays, and the enzyme activity of glyoxal oxidase. An alternative reductant, catechol, proved more desirable as it is not an inhibitor for any of the assays described herein. As an alternative to the foregoing, ascorbate may be used in the PAM-catalyzed reaction, but later removed (e.g., by deactivating with ascorbate oxidase) prior to assaying for glyoxylate.

In one preferred embodiment, the invention contemplates searching for new previously-undocumented or unstudied amidated hormones, amidated fatty acids or other natural amidated products of the PAM reaction (or the products of PHM and PAL-catalyzed reactions or of PHM and a Lewis base). The searches aid the determination of the existence of PAM (or PHM) activity or PAM (or PHM) substrates (which are usually compounds having glycine residues in free acid form and attached to a carbonyl group). In some preferred embodiments cells suspected of such enzymatic activity and/or of providing such substrates are incubated, preferably in the presence of an inhibitor of PAM to cause a build up of the glycine-containing precursor. In some embodiments, a secretagogue is utilized to better cause secretion of small molecules into the culture media. The media may then be harvested, with cells being removed. The media is then fractionated with each fraction being tested by one or more of the techniques discussed herein for the presence of glycine-extended precursors. These tests preferably add PAM (or a combination of PHM and PAL, or a combination of PHM and Lewis base) under conditions that convert glycine-extended precursor into amidated product and glyoxylate. Assays for glyoxylate, when positive, then provide evidence of the relevant glycine-extended precursors in the fraction being tested. Any assay for a glyoxylate may be used. However, it is preferred to use assays as taught herein wherein glyoxylate is converted either directly or indirectly into, inter alia, hydrogen peroxide, followed by assaying for hydrogen peroxide. Preferably, such hydrogen peroxide assays are the luminescent or fluorescent techniques taught herein. Fractions testing positive for glyoxylate are likely to have the glycine-extended precursors of interest. PAM activity and the creation of amidated products is also likely. The amino acid sequence in most of families of peptide hormones have loose homology and certain patterns can be recognized upon further analysis of the fractions that test positive for glyoxylate. New peptide hormones are thereby identified for further study and are likely to have pharmaceutical importance. Peptide hormones frequently perform functions that are essential to survival.

To further confirm that glyoxylate identified in particular fractions is a result of the activity of PAM or PHM, the foregoing steps may be repeated for a fraction that has previously tested positive, but without adding PAM (or PHM+PAL, or PHM+Lewis base). If much lower levels of glyoxylate are then detected, this provides further confirmation that the larger amount of glyoxylate originally detected results from PAM or PHM reactions.

To further verify that the glycine-extended compounds identified in fractions that test positive for glyoxylate, are in fact precursors of the amidated hormones and other amidated compounds that are sought, these compounds can be further analyzed for additional indicia that they are in fact likely to be PAM or PHM substrates. For example, tissue distribution of MRNA or cDNA is relevant. Significantly different levels of expression in different tissues is evidence of a desirable compound. Amino acid sequences terminating in C-terminal glycine is an easily-recognizable characteristic. The DNA which gives rise to such peptides identified in accordance with the invention may be analyzed, and desirably includes a signal region in reading frame with a peptide coding region that either terminates with C-terminal glycine or has glycine followed by single or multiple base amino acids that serve as post-translational cleavage sites immediately downstream.

Glyoxylate Assays

The glyoxylate produced during the amidation reaction can be assayed by a variety of techniques. Spectrophotometric enzyme-linked assays for glyoxylate may be initiated by the addition of malate synthase and malate dehydrogenase. The assay may contain 10 mM TEA-HCl pH 7.8, 150 μM/8.25 μM MTS/PMS, 10 mM MgCl₂, 400 μM acetyl-CoA, 500 μM NAD⁺, 0-50 μM glyoxylate, 6 units/ml malate synthase, and 6 units/ml malate dehydrogenase in a final volume of 1 ml. The absorbance at 490 nm may be measured after one hour incubation at 37° C. in the dark. The small amount of MTS reduced for the zero glyoxylate control should be subtracted from that obtained in the presence of glyoxylate.

Chemical Production of Glyoxylate

Glyoxylate is a product of base-catalyzed N-dealkylation of carbinolamides. Incubation of 2.5 mM α-hydroxyhippurate in 1.0 M NaOH for 12 hours at 37° C. may result in the conversion of α-hydroxyhippurate to benzamide as determined by reverse-phase HPLC. The resultant glyoxylate concentration may be determined via the malate synthase/malate dehydrogenase couple after appropriate dilution with H₂O to a final glyoxylate concentration of <40 μM.

Enzymatic Production of Glyoxylate

Hippurate (N-benzoylglycine, C₆H₅-CO—NH—CH₂-COO⁻) is a PAM substrate that is oxidatively converted to benzamide and glyoxylate. Hippurate oxidation at 37° C. may be initiated by the addition of peptidylglycine α-amidating monooxygenase, (0.6 mg) 100 mM MES pH 6.0, 2.0 μM Cu(NO₃)₂, 10 mM ascorbate, and 3.5 mM hippurate in a final volume of 0.5 ml. At 10 min intervals over a period of 110 minutes, 45 μl aliquots may be removed and added to 10 μl of 6% (v/v) trifluoroacetic acid to terminate the reaction. Percent conversion of hippurate to benzamide may be determined at each time interval by reverse-phase HPLC.

Approximately 20 nanomoles of glyoxylate may be removed from the HPLC vials and added to a 0.9 ml solution that contain necessary components for the glyoxylate assay excluding the enzyme couple and MTS/PMS. Ascorbate may be eliminated from all samples, prior to glyoxylate determination, with 10 min incubation in the presence of 12 units of ascorbate oxidase at 37° C. Following ascorbate removal the addition of 100 μl enzyme couple and PMS/MTS may bring the assay to a final volume of 1 ml which contains 100 mM TEA-HC1 pH 7.8, 10 MM MgCl₂, 400 μM acetyl-CoA, 500 μM NAD⁺, 6 U/ml malate synthase, 6 U/ml malate dehydrogenase, and 12 units ascorbate oxidase. The glyoxylate concentration may be determined by measuring the absorbance increase at 490 nm after incubation at 37° C. for 1 hr. The amount of glyoxylate and benzamide produced in one such study are shown in Table 1 below. A control for this experiment was performed at each time point, and contained no hippurate.

TABLE 1
Ratio of [Glyoxylate] Produced to [Benzamide] Produced
by the PAM Treatment of Hippurate

| | Glyoxylate Produced | Benzamide Produced | [Glyoxylate]/ |
|---|---|---|---|
| Time | (mM) | (mM) | [Benzamide] |
| 40 | 0.69 | 0.58 | 1.2 |
| 50 | 0.67 | 0.70 | 0.96 |
| 60 | 0.71 | 0.80 | 0.89 |
| 70 | 0.75 | 0.90 | 0.83 |
| 80 | 0.77 | 0.98 | 0.79 |
| 90 | 1.3 | 1.1 | 1.2 |
| 100 | 1.3 | 1.2 | 1.1 |
| 110 | 1.3 | 1.3 | 1.0 |

Average ± standard deviation = 1.0 ± 0.16
Note:
Reactions were initiated by the addition of PAM to 2.5 mM hippurate. At the indicated time, an aliquot was removed and assayed for benzamide by HPLC and glyoxylate using the malate synthase/malate dehydrogenase/MTS/PMS system.

Glyoxylate can also be assayed for by its conversion to oxalate and hydrogen peroxide by the action of glycolate oxidase or glyoxal oxidase. All glycolate oxidase reactions were performed in Phosphate Buffer pH 7.8, 0.1 mM FAD (fluorescent assay), or 0.1 mm FMN (luminescent assay) with 0.48 U/assay of enzyme. The glyoxal oxidase reaction was carried out at pH 8.0 in 100 mM TEA Buffer with 1 U/ml HRP. Other details are outlined in the figures.

Screening and Identification of α-amidated Peptide Mouse At-t20 cells, known to secrete mouse Joining Peptide-Gly (mJP-Gly), were grown in the appropriate cell culture medium to 70% confluency, cells were collected and resuspended in fresh medium containing 2 μM disulfiram, a known PAM inhibitor, and incubated for 15 hours for accumulation of mJP-Gly. Spent medium was collected, acid extracted with 0.1% TFA, and desalted prior to RP-HPLC analysis. The desalted extract was lyophilized and resuspended in 200 μl of 0.1% TFA/0.001% Triton-X, prior to HPLC analysis. 50 μl aliquots were injected onto a C18 RP-HPLC column equipped with a quaternary solvent delivery system. Peptides were separated with a gradient elution of 0.1% TFA/ACN over the timespan of 65 minutes at a flow rate of 1.0 ml/min. Samples containing the internal standard were spiked with 2.5 nanomoles mJP-Gly prior to RP-HPLC separation. One minute fractions were collected over the 65 minute separation, lyophilized and treated with PAM. PAM condition utilized were 40 mM MES pH 6.3, 1 mM Ascorbate/Catechol, 0.5 μM CuSO4, 50 U/ml HRP, 15 U/ml PAM the reaction was carried out in a volume of 300 μl for 3 hours at 398 K. Fractions were then brought to 600 μl in 100 mM Sodium Phosphate pH 7.8, and 0.24 u/ml Glycolate Oxidase was added, the reaction persisted for one hour at 398 K. Luminescent detection was carried out utilizing 1 mM Luminol, 1 mg/ml HRP, in Sodium Carbonate Buffer pH 10.5. Fractions whose fate was to be tested by MALDI-TOF were simply lyophilized after RP-HPLC separation, resuspended in 200 μl 0.1% TFA and analyzed.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

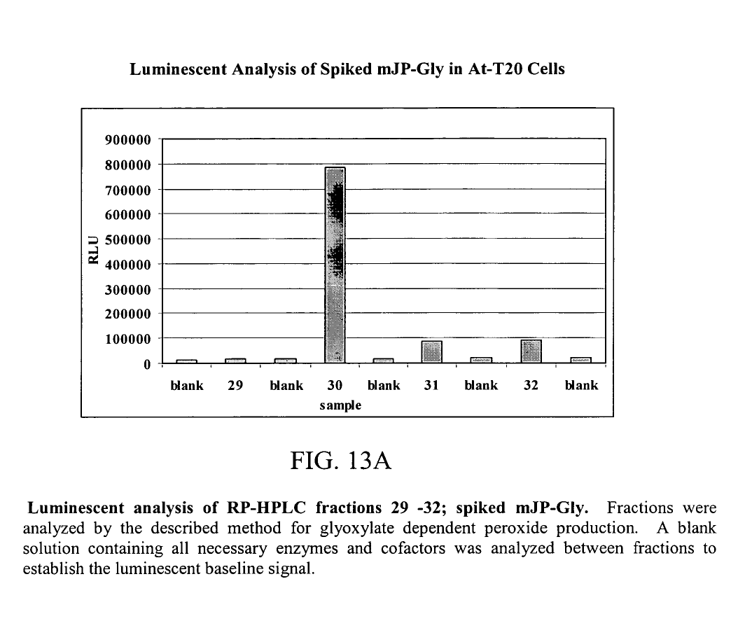

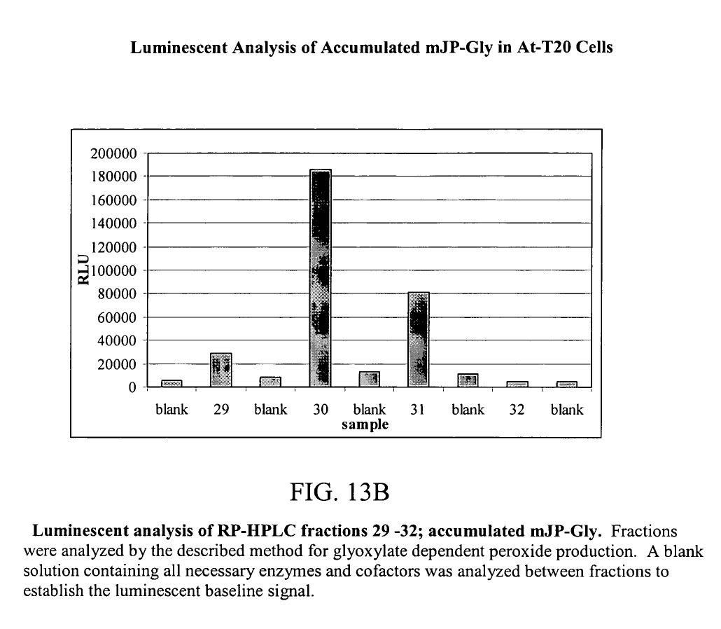

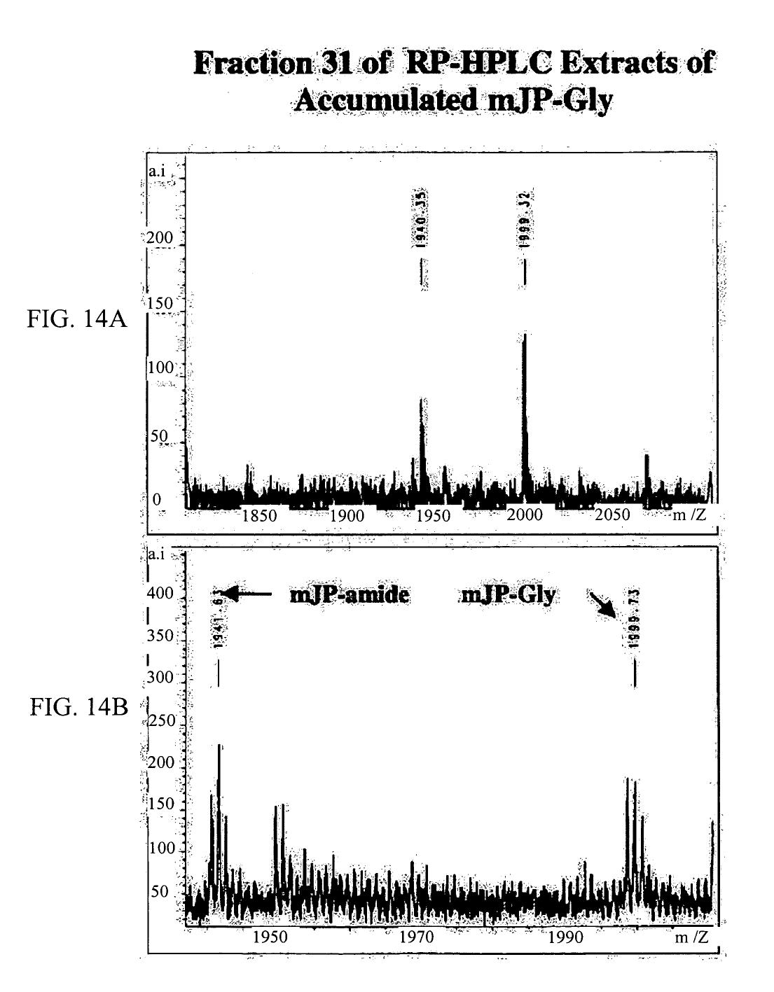

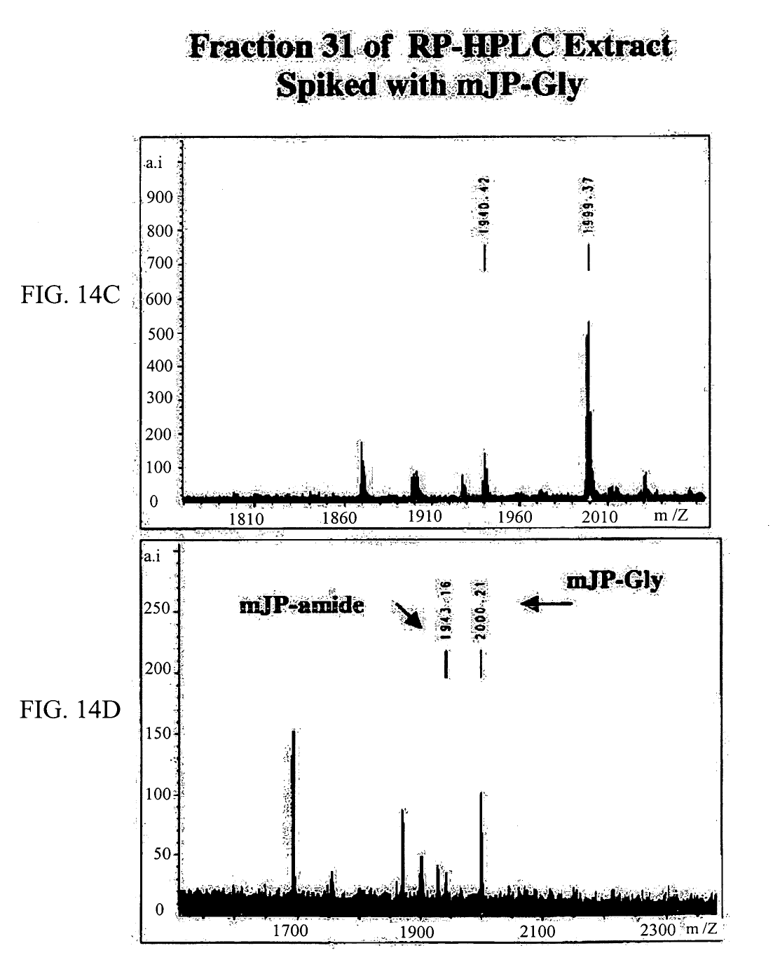

Based on sensitivity alone the luminescent enzyme assay utilizing glycolate oxidase was chosen for application of the glyoxylate assay as a route to the identification of an α-amidated peptide. Any of the assays of the present invention could be used; however, the most sensitive of these techniques is more desirable. A cell line of mouse pituitary cells known to express mouse joining peptide (mJP) (Ala-Glu-Glu-Glu-Ala-Val-Trp-Gly-Asp-Gly-Ser-Pro-Glu-Pro-Ser-Pro-Arg-Glu-Gly) were grown in cell culture to approximately 80% confluency. Cells were then grown in the presence of a PAM inhibitor in order to accumulate the glycine-extended peptides. Spent media was fractionated by Reverse-Phase High Performance Liquid Chromatography (RP-HPLC), and each fraction was then treated with PAM. Fractions positive for glyoxylate were analyzed against a sample containing an internal standard of mJP, to conclude the glyoxylate assay was indeed correct at identifying the presence of a glycine-extended/α-amidated peptide. To further the analysis, a separate set of fractions which did not undergo the PAM reaction were analyzed by MALDI TOF Mass Spectrometry for the presence of the mJP. Demonstration of the glycine-extended form of mJP being present in the same fraction as glyoxylate proves that both the cell culture PAM reaction was indeed inhibited thus allowing the formation of glyoxylate upon treatment with PAM. All data demonstrated that glyoxylate, mJP-Gly, and mJP-amide were present in the same fraction (see FIGS. 13A-13B and 14A-14D).

### EXAMPLE 2

### Detection of Rat CGRP-Gly in CA-77 Cell Conditioned Medium Using the Enzyme-Linked Luminescent Assay

Rat CA-77 cells were grown in T75 flasks to near confluence (60-90%) in DMEM:F10 (supplemented with insulin, transferring and selenium) and 10% fetal bovine serum (FBS). Cells were washed twice with PBS to remove residual FBS. Cells were grown further for either 24 or 48 hours in DMEM:F10 (supplemented with transferring and selenium) in the presence of a secretagogue, 0.1 μM dexamethasone, and a PAM inhibitor, 100 μM diethyldithiocarbamic acid. Insulin, which is present at high concentration, was omitted from the second growth medium to avoid potential interference during purification. Conditioned medium was harvested and cellular debris was removed by centrifugation.

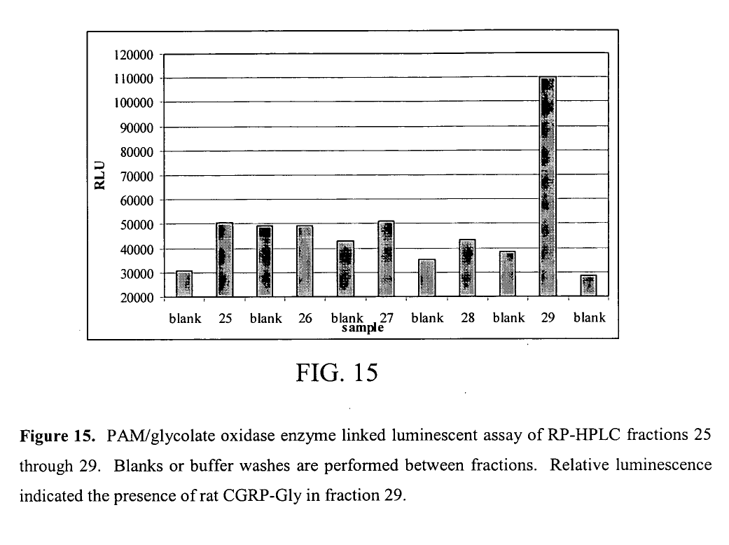

Approximately 270 mL of conditioned medium was loaded onto an Amberchrom CG300M reversed-phase column (1.1 cm×14.5 cm) equilibrated with 0.1% TFA, 2% MeCN. The column was operated at 180 cm/hr and the absorbance of the column effluent was monitored at 280 nm. The column was washed with 0.1% TFA, 10% MeCN to remove cell culture by-products. The column was washed with 0.1% TFA, 50% MeCN and the resultant peak, which had previously been shown to contain the majority of the peptides in the conditioned medium, was collected. The 50% MeCN fraction was concentrated approximately 10-fold by lyophilization. Acetonitrile was added back to the concentrated fraction to bring the final concentration to approximately 5%. Any insoluble material present following the acetonitrile addition was removed by centrifugation. The concentrated fraction was subjected to further purification by RP-HPLC to fractionate peptides. The concentrated fraction was loaded onto a Hypersil BDS C18 column (4.6 mm×250 mm) equilibrated with 0.1% TFA, 20% MeCN. The column was operated at 1.2 mL/min. and the absorbance of the column effluent was monitored at 220 nm. The column was washed with 0.1% TFA, 20% MeCN for 18 minutes, followed by a linear gradient from 0.1% TFA, 20% MeCN to 0.1% TFA, 52% MeCN over 60 minutes. Fractions, approximately 1.5 mL each, were collected across the entire gradient. Fractions 15 through 41 were collected and concentrated to dryness by lyophilization. Fractions were screened using the PAM/glycolate oxidase enzyme linked luminescent assay for the presence of rat CGRP-Gly. A strong luminescent signal was detected in fraction number 29; this fraction was co-incident with the retention time for rat CGRP-Gly reference standard, which had previously been spiked into conditioned medium and purified chromatography under conditions identical to those described above (FIG. 15).

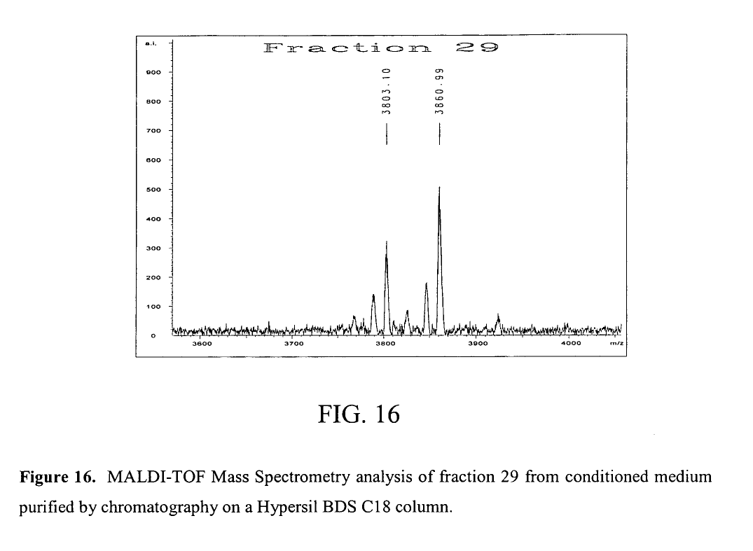

A second experiment was performed where the presence of rat CGRP-Gly in the conditioned medium was corroborated by MALDI-TOF MS. Approximately 100 mL of conditioned medium was loaded onto an Amberchrom CG300M reversed-phase column (1.1 cm×14.5 cm) equilibrated with 0.1% TFA, 2% MeCN. The column was operated at 180 cm/hr and the absorbance of the column effluent was monitored at 280 nm. The column was washed with 0.1% TFA, 10% MeCN remove cell culture by-products. The column was washed with 0.1% TFA, 50% MeCN and the resultant peak was collected. The 50% MeCN fraction was concentrated approximately 10-fold by lyophilization. Acetonitrile was added back to the concentrated fraction to bring the final concentration to approximately 5%. Any insoluble material present following the acetonitrile addition was removed by centrifugation. The concentrated fraction was subjected to further purification by RP-HPLC to fractionate peptides. The concentrated fraction was loaded onto a Hypersil BDS C18 column (4.6 mm×250 mm) equilibrated with 0.1% TFA, 20% MeCN. The column was operated at 1.2 mL/min. and the absorbance of the column effluent was monitored at 220 nm. The column was washed with 0.1% TFA, 20% MeCN for 18 minutes, followed by a linear gradient from 0.1% TFA, 20% MeCN to 0.1% TFA, 52% MeCN over 60 minutes. Fractions, approximately 1.5 mL each, were collected across the entire gradient. Fractions 26 through 34 were collected and concentrated to dryness by lyophilization. Fractions 26-34 were subjected to MALDI-TOF MS. Both rat CGRP-Gly and CGRP amide were detected in Fraction 29 (FIG. 16); these data are consistent with the molecular mass observed for the rat CGRP-Gly reference standard (3,860.8 Da) and the predicted molecular mass for rat CGRP amide (3,803.1 Da); amidation leads to a loss of 58 Da. Furthermore, fraction 29 was co-incident with the luminescent signal previously identified using the enzyme-linked assay and the established retention time for rat CGRP-Gly reference standard.

Fluorescent Assays for the Detection of Hydrogen Peroxide

Several fluorophores exist for the fluorescent determination of hydrogen peroxide generated in solution. Fluorescence dyes for hydrogen peroxide determination have been designed as substrates for the enzyme horseradish peroxidase. For example, horseradish peroxidase (E. C. 1.11. 1.7.) utilizes Amplex Red ® as an electron donor in a disproportionation reaction of hydrogen peroxide to water and molecular oxygen. Amplex red becomes oxidized to the intensely fluorescent compound resorufin, in a stoichiometric ratio to hydrogen peroxide consumption. Amplex red can be utilized in a continuous assay format for enzymatic activity assays which produce stoichiometric quantities of hydrogen peroxide. Amplex Red® has a quantum yield (Φ) at pH=9 (Φ=photons absorbed/photons emitted) of 0.75, rendering Amplex Red® a highly fluorescent fluorophore.

Resorufin fluorescence is typically measured with an excitation λ of 560 nm and emission λ of 589 nm. The sensitivity of Amplex Red, like all fluorophores, can be compromised in a biochemical assay format as a result of a high signal/noise ratio. Fluorescence is a ‘dark’ process allowing for greater sensitivity as result of the stokes shift. The Stokes shift describes the distance of the red shift (near IR) exhibited by a fluorophore from a shorter excitation wavelength to the longer emissive wavelength. Quantification of the emission generated by a fluorophore measures an observable event from dark (no light) to bright. In particular, biochemical based assays are compromised for the most part by the auto-oxidation of amplex red.

Fluorescent Assays for Glyoxylate

The Flavin Dependent Glyoxylate Consuming Enzyme Glycolate Oxidase

Glycolate oxidase (hydroxyacid oxidase, E. C. 1. 1.3.15) is an oxidoreductase enzyme that typically catalyzes the oxidation of a primary alcohol in the presence of O₂ to a ketone and hydrogen peroxide). Glycolate oxidase is a flavoenzyme, such enzymes require a flavin prosthetic group for electron transfer from donor to acceptor. Classically, glycolate oxidase has been known as a FMN (flavin mononucleotide) dependent flavoprotein, however published data provides evidence for the ability of this enzyme to be catalytically competent in the presence of alternate flavin prosthetic groups, namely, FAD. The flavin-binding domain of glycolate oxidase is deeply seated within the interior of the enzyme, and tightly bound to the interior flavin binding domain with dissociation constants of 10⁻⁸ to 10⁻¹⁰ M. Riboflavin is the simplest of all the flavins in that it contains the basic isoalloxazine “flavin” domain and a D-ribitol group. FMN is riboflavin with a free phosphate attached to the D-ribitol, and, lastly, FAD is a conjugate of FMN containing an AMP moiety via a phospho-diester bond).

Utilization of Glycolate Oxidase as a Fluorescent Assay for Glyoxylate

Fluorescence assays are limited in most part by signal/noise, due to the spontaneous oxidation (or reduction) of fluorophores resulting in significant background fluorescence. In addition, fluorescence detection can be further compromised in biological samples by the intrinsic fluorescence of biological molecules (peptides, proteins, etc), thus further affecting signal/noise. In the presence of FMN, Amplex Red non-enzymatically oxidizes to resorufin to the extent that glycolate oxidase activity cannot be measured via an HRP-dependent Amplex Red assay. However, non-enzymatic oxidation of Amplex Red is significantly reduced in the presence of FAD. FAD is a cofactor that completely supports glycolate oxidase activity. In order to link glycolate oxidase activity to an HRP/Amplex Red based assay, the cofactor must be FAD.

The Lignolytic Degrading Enzyme Glyoxal Oxidase

Glyoxal oxidase (GLOX) is a basidiomycete fungal enzyme, one of the three enzymes produced for the degradation of lignin. The fungal lignin degradation pathway plays a major role in the decomposition of detritus, an integral part of the global carbon cycle. Lignin is the second most abundant substance on the planet second only to cellulose, and forms the “woody” tissue of plants. Collectively, lignin is comprised of several monolignols, namelyp-courmaryl, sinapyl, and coniferyl alcohols which compromise the basic set of monomers for the lignin polymer. Degradation of lignin is imperative to the regeneration of carbon, eventually producing atmospheric CO₂. The catalytic role of glyoxal oxidase is the oxidation of aldehydes to carboxylic acids coupled to the concomitant reduction of dioxygen to hydrogen peroxide. GLOX is a copper metalloenzyme, containing a free radical-coupled copper active site. The radical-copper catalytic motif comprises the two-electron redox active site. More importantly, GLOX is isolated in the reduced form. Activation of the reduced enzyme requires oxidation via treatment with a strong oxidant such as Ir (IV) or Mo (V), or the presence of Lignin peroxidase (LiP) or horseradish peroxidase (HRP) for catalytic activity.

Utilization of Glyoxal Oxidase for the Quantification of Glyoxylate

Glyoxal oxidase has broad substrate specificity among simple aldehydes. Methylglyoxal the preferred substrate has a Kₘ=0.64 mM as compared to glyoxylic acid Kₘ=2.5 mM, and a kcat/KM ratio of 12.4% the activity for glyoxylate compared to methylglyoxal. Moreover, GLOX has an acidic pH optimum which when applied to the Amplex Red detection system, compromises detection limits because the reduced Φ_{resorufin} at pH 6.0 from 0.75 to 0.11, an overall 85% Φ_{resorufin} reduction at pH 6.0.

GLOX functions in the detection system of glyoxylate like glycolate oxidase: the oxidation of the glyoxylate gem diol to oxalate with the concomitant production of H₂O₂. H₂O₂ production is coupled to Amplex Red oxidation to resorufin as catalyzed by HRP.

GLOX is isolated in the catalytically inactive, reduced form requiring the presence of an oxidant for catalysis. However, the use of strong oxidants must be avoided as this results in the spontaneous oxidation of Amplex Red. Thus, HRP has a dual role in this detection system as it both activates the reduced glyoxal oxidase, and converts Amplex Red to resorufin via a stoichiometric reaction with H₂O₂.

Chemi-Luminescent Assays for Hydrogen Peroxide

Chemi-luminescence is a similar process to that of fluorescence however excitation to the excited state is dependent upon a chemical reaction rather than incident light (λₑₓ i.e. fluorescence).

Fluorescence and chemi-luminescence, undergo excitation, internal conversion, followed by an observable red shift emission. Internal conversion is a non-photo-emissive transition of electrons between two states of the same spin number, and relates to the quantum yield of a fluorophore (Φ). Phosphorescence also undergoes a non-emissive transition, however initially it is between two different spin states. This type of non-emissive transfer is described as intersystem crossing and is followed by internal conversion. Chemi-luminescence has been utilized for biochemical applications much like florescence due to its enhanced sensitivity, on account that it is also a dark process.

Luminol in the presence of an oxidative (1 or 2 e⁻ oxidant) metal catalyst such as Mn (II) [Cu(phen)₃²⁺, HRP; (Fe(II)), K₄Fe(CN)₆.3H₂O; (Fe(II)) [110], and Co(II) and hydrogen peroxide, becomes chemically excited to a triplet state. Excitation is followed by internal conversion to a singlet state, and lastly photo-emission. The amount of luminol photo-emission is proportional to the concentration of hydrogen peroxide present. Luminol prior to becoming excited must be in the doubly de-ionized form requiring that luminol chemi-luminescence be carried out in a basic environment. Of all possible metal oxidants it was found that use of HRP resulted in the greatest light emission by luminol.

Materials and Methods Used in The Fluorescent and Luminescent Assays Infra

Materials

Amplex red, resorufin, and luminol were purchased from Molecular Probes, (Eugene, OR); glycolate oxidase, HRP, FMN, FAD, MES, sodium glycolate, N-dansyl-Tyr-Val-Gly, were purchased from sigma-aldrich; recombinant rat PAM was a gift from Unigene Labs, Inc. (Fairfield, N.J.); and glyoxal oxidase (source: Phanerochaete chryosporium) was a gift from Dr. James Whittaker (OGI School of Science and Engineering, Oregon Health and Science University, Beaverton, Oreg.). Black flat-bottom, and U-shaped well plates were purchased from Corning. All other reagents were of the highest quality commercially available.

Methods

Standardization of the Fluorophore Resorufin

A standard solution of resorufin was initially used to standardize the micro-plate fluorometer for resorufin fluorescence. Concentrations ranging from 30 nM to 9 μM of were analyzed for fluorescent response at λₑₓ=30 nm and λₑₘ=584 nm to generate a resorufin standard curve. Samples were analyzed in black U-shaped microplates, in a Fluoroskan II microplate reading fluorometer equipped with the MTX software analysis package.

Standardization of H₂O₂ Produced Fluorescence

A standardized hydrogen peroxide solution was utilized for the preparation of a hydrogen peroxide standard curve for resorufin production. Concentrations ranging from 30 nM to 9 μM peroxide were analyzed in a solution containing, HRP (1 U/ml) and 50 μM Amplex Red in 50 mM phosphate buffer pH 6.0, and pH 8.0.

Glyoxal Oxidase (GLOX ) Fluorescent Assay for Glyoxylate

Standardization of the Fluorescent GLOX Assay with Standard Methyl Glyoxal and Glyoxylate

The GLOX assay consisted of a standard solution of 50 mM sodium phosphate pH 6.0, 50 μM Amplex red, HRP (1U/ml), and either 0.03-2.3 μM methyl glyoxal or 0.3-1.7 μM glyoxylate. The reaction was initiated by the addition of GLOX (final concentration=0.4 mg/ml) and fluorescence was determined after 1 hour at 37° C. in the dark for the glyoxylate substrate and after a 30 min 37° C. incubation for methyl glyoxal (λₑₓ=530 nm and λₑₘ=584 nm). The fluorescence produced from Amplex red oxidation in the absence of glyoxylate or methyl glyoxal was subtracted from that obtained in the presence of glyoxylate/methyl glyoxal.

Standardization of the Fluorescent Glyoxal Oxidase Assay for PAM Produced Glyoxylate

Glyoxylate production was initiated by the addition of PAM (15 U/L) to a solution containing 40 mM MES/NaOH pH 6.0, 10 U/ml HRP, 1.0 mM catechol, 0.5 μM CU(SO₄), 20 μM dansyl-Tyr-Val-Gly, the reaction proceeded for one hour at 37° C. It is necessary to note that catechol was used as the reductant to support PAM catalysis, as the Fenton chemistry produced by ascorbate in the presence of copper (generation of OH, and H₂O₂) resulted in the complete auto-oxidation of Amplex Red. The complete PAM dependent conversion of 20 μM N-dansyl-Tyr-Val-Gly to N-dansyl-Tyr-Val-NH₂ and glyoxylate was verified by RP-HPLC to quantify the exact concentrations of [dansyl-Tyr-Val-NH₂] and [glyoxylate] (100% conversion, 20 μM N-dansyl-Tyr-Val-NH2 and glyoxylate). Aliquots of the PAM produced glyoxylate pertaining to variable concentrations (0.3, 0.7, 1.6, and 2.3, μM) of were utilized for analysis by the GLOX assay. Aliquots of glyoxylate were added to a solution of 50 mM sodium phosphate pH 6.0, 50 μM Amplex red, at HRP (1 U/ml) and a final concentration of 0.4 mg/ml GLOX. All samples were incubated for 1 hour at 37° C. for one hour.

Glycolate Oxidase (GO) Fluorescent Assay for Glyoxylate

Standardization of the Fluorescent Glycolate Oxidase (GO) Assay with Glycolate and Glyoxylate.

The glycolate oxidase assay consisted of a standard solution of 70 mM sodium phosphate pH 7.8, 50 μM Amplex red, 0.1 mM FAD, HRP (1 U/ml), and either 0-10 μM glycolate, or 0-10 μM glyoxylate in a final volume of 300 μl. Commercially available glycolate oxidase as purchased from sigma contains 2mM FMN. Excess FMN was removed from the enzyme solution by dialysis against 3 L of 10 mM Tris-HCl pH 7.4. 0.75 mM FAD was then added to the apo-enzyme, and pre-incubated for at least 1 hour in order to be fully catalytically competent in the glyoxylate assay. It is necessary to note that the enzyme must be pre-incubated with the FAD cofactorprior to its use. The reaction was initiated by the addition of glycolate oxidase (final concentration=0.2 mg/ml) and fluorescence determined after 1 hour at 37° C. for the glyoxylate substrate, and after a 20 min. incubation for the glycolate substrate. Fluorescence produced from the Amplex red oxidation in the absence of glyoxylate or glycolate was subtracted from that obtained in the presence of glyoxylate/glycolate.

Standardization of the Fluorescent Glycolate Oxidase (GO) Assay for PAM Produced Glyoxylate.

Glyoxylate production was initiated by the addition of PAM (15 U/L) to a solution containing 40 mM MES/NaOH pH 6.0, 10 U/ml HRP, 1.0 mM catechol, 0.5 μM Cu(SO₄), 20 μM dansyl-Tyr-Val-Gly, the reaction proceeded for one hour at 37° C. The PAM dependent conversion of 20 μM N-dansyl-Tyr-Val-Gly to N-dansyl-Tyr-Val-NH₂ and glyoxylate was verified to be 100% by a RP-HPLC assay, to ensure a 20 μM PAM produced glyoxylate solution. Aliquots of the PAM produced glyoxylate pertaining to variable concentrations (1.0, 3.0, 5.0, 7.0 and 9.0 μM) were taken for analysis by the glycolate oxidase assay. The aliquots of glyoxylate were added to a solution of 70 mM sodium phosphate pH 7.8, 50 μM Amplex red, and HRP (1 U/ml) in a final volume of 300 μl. All fluorescent reactions were initiated by the addition of 0.2 mg/ml GO, 0. 1 mM FAD (final concentration). All samples were incubated for 1 hour at 37° C. prior to fluorescent analysis of resorufin production.

Chemi-Luminescent Assays for Glyoxylate

A Hydrogen Peroxide Chemi-Luminescent Standard Curve

A standard working solution of 0.1 mM Luminol in 0.1 M NaHCO₃ pH 10.5 was prepared and purged in an atmosphere of N₂, and stored at 4° C. in the dark, the stock was prepared fresh daily. Black flat-bottom microplates were prepared by the addition of 8 μl HRP (11 mg/ml) and 67 μl of sodium bicarbonate (0.4 M), microplates were incubated for at least 45 min. in the dark prior to luminetric analysis. A Berthold/Tropix TR-717 binary injector microplate luminometer, with a 500 μl dead volume was utilized for all microplate luminescent assays. The luminometer dead volume is defined as the volume of sample retained in-line after injection prior to reaching the detector for measurement. The primary injector was programmed to inject 200 μl of the sample (hydrogen peroxide) and the offset injector was programmed to inject 25 μl of the luminol stock. Thus, each well contained a final concentration of 0.3 μg/μl HRP, 8.0 μM Luminol, 0.1M NaHCO₃ pH 10.5, in a final volume of 300 μl.

Standard hydrogen peroxide concentrations (5 nM, 7 nM, 10 nM, 20 nM, 50 nM) were prepared within a 600 μl final sample volume for luminetric analysis. Microplates were prepared such that the injection of 200 μl peroxide, 25 μl luminol, in addition to the HRP/NaHCO₃ prepared plates resulted in a final well volume of 300 μl. Luminetric measurements (RLU=Relative Luminescence) were obtained in the flash kinetic mode, with a 1.6 sec. time delay between sample and luminol injection.

All measurements were obtained immediately following luminol injection for a time duration of 10 seconds. Three sequential 200 μl injections of the standard were necessary to analyze the total 600 μl hydrogen peroxide sample. A water blank control was injected prior to and in between each standard H₂O₂ solution in order to define a baseline background RLU luminescent signal, for signal/noise analysis. A standard curve was generated from the total RLU generated per standard peroxide solution.

Chemi-Luminescent Glycolate Oxidase Assay with Glyoxylate

Following standardization of the chemi-luminescent dependent system for the quantification of hydrogen peroxide, the detection system was applied to the quantification of glyoxylate dependent H₂O₂ production. A standard glyoxylate solution (7 nM, 20 nM, 50 nM, 80 nM, 200 nM) was utilized to develop a standard curve for the detection of glycolate oxidase produced hydrogen peroxide. All glycolate oxidase reactions were performed in 100 mM phosphate buffer pH 7.8, with a final concentration of 0.48 U/ml glycolate oxidase, 0.2 mM FMN in a final volume of 600 μl. It was not necessary to dialyze the FMN containing enzyme as FMN does not interfere with the chemi-luminescent detection system. Microplates were prepared with 8 μl HRP (11 mg/ml) and 67 μl of sodium bicarbonate (0.4 M), incubated for one hour, prior to the analysis of glyoxylate dependent H₂O₂ production. Each 600 μl reaction was injected over three wells as 200 μl aliquots per well. In total, the addition of sample (200 μl), luminol (25 μl), HRP (8 μl) and NaHCO₃ (67 μl), resulted in a final concentration per well of 0.3 μg/μl HRP, 8.0 μM Luminol, 0.1M NaHCO₃ pH 10.5, at a final volume of 300 μl. The background RLU was determined by the addition of all reagents excluding the glyoxylate, and the blank was injected prior to and between each standard glyoxylate concentration. A standard curve was generated by the addition of RLU response per well for each standard glyoxylate sample. The detection limit was defined as the signal over background which fit a linear regression of RLU vs. [glyoxylate].

Chemi-Luminescent Glycolate Oxidase Assay for PAM produced Glyoxylate

Preparative RP-HPLC for the Collection of the PAM substrate N-dansyl-Tyr-Val-Gly; An Empirical Trial for the Use of the Platform Technology to Detect a Glycine-Extended Peptide

A 20 μM sample of the PAM substrate N-dansyl-Tyr-Val-Gly was injected onto a Keystone ODS Hypersil C₁₈ column (100×4.6 mm, 5 μ particle size) RP-HPLC column, equipped with a Bio-Rad Model 1200 in-line fraction collector at 1.0 min intervals. The analyte was separated and collected by an isocratic mobile phase of 100 mM sodium acetate pH 6.0/acetonitrile (52/48) at flow rate of 1.015 ml/min [90]. Fractions were collected over the 4 minute separation, lyophilized to complete dryness, and reconstituted in PAM assay conditions consisting of 40 mM MES/NaOH pH 6.3, 1 mM sodium ascorbate, 0.5 μM CuSO₄, 10 U/ml HRP, 0.015 U/ml PAM in a final volume of 300 μl. The PAM reaction proceeded for 1 hour at 37° C., 10 μl aliquots per fraction were removed for percent conversion analysis of N-dansyl-Tyr-Val-Gly to N-dansyl-Tyr-Val-NH₂ by the described RP-HPLC assay [111]. Conversion of the N-dansyl-Tyr-Val-Gly substrate to the products N-dansyl-Tyr-Val-NH₂ and glyoxylate, was analyzed by a RP-HPLC PAM activity assay, to ensure 100% conversion of the substrate in order to verify the reaction products at [N-dansyl-Tyr-Val-NH₂] =[glyoxylate] =20 μM. Following the PAM reaction, and product analysis, an aliquot of 2 U/ml of ascorbate oxidase was added to all PAM reactions at 40 mM MES pH 6.3 to oxidize all remaining ascorbate. It was necessary to oxidize all remaining ascorbate in the PAM reaction, as the reductive properties of ascorbate suppress the oxidation of luminol, necessary for light production. The resultant solution was utilized as a standard PAM produced glyoxylate stock, for use in the glycolate oxidase assay for PAM produced glyoxylate.

Aliquots of the PAM reaction corresponding to variable concentrations of glyoxylate (7 nM, 20 nM, 80 nM, 200 nM) were taken for analysis by the chemi-luminescent glycolate oxidase assay. Aliquots were added to a solution containing 100 mM sodium phosphate buffer pH 7.8, reactions were initiated by the addition of 0.48 U/ml glycolate oxidase, 0.2 mM FMN and reacted for one hour at 37° C. at a final volume of 600 μl. The RLU response per [glyoxylate] sample was then analyzed.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A method for detecting the presence of glyoxylate in a test sample comprising the steps of:
(A) contacting said test sample with one or more agents that convert glyoxylate, directly or indirectly, to hydrogen peroxide as one reaction product; and
(B) assaying for the presence of hydrogen peroxide.

2. The method of claim 1 wherein hydrogen peroxide is detected by a fluorescent assay.

3. The method of claim 2 wherein said fluorescent assay comprises reacting a fluorophore with a sample suspected of containing hydrogen peroxide to form a peroxide-dependent fluorescent product when hydrogen peroxide is present.

4. The method of claim 3, wherein said fluorophore is amplex red and said fluorescent product is resorufin.

5. The method of claim 1 wherein said conversion of glyoxylate to hydrogen peroxide is performed enzymatically.

6. The method of claim 4 wherein said conversion of glyoxylate to hydrogen peroxide is performed in the presence of glyoxal oxidase.

7. The method of claim 4 wherein said conversion of glyoxylate to hydrogen peroxide is performed in the presence of glycolate oxidase.

8. The method of claim 1 wherein hydrogen peroxide is detected by a luminescent assay.

9. The method of claim 8 wherein said luminescent assay comprises reacting luminol with a sample suspected of containing hydrogen peroxide.

10. The method of claim 9 wherein luminol is reacted at basic pH and in the presence of an oxidative catalyst.

11. The method of claim 10 wherein said oxidative catalyst is horseradish peroxidase.

12. The method of claim 9 wherein luminol is reacted at a pH no lower than 10.5.

13. The method of claim 8 wherein said conversion of glyoxylate to hydrogen peroxide is performed enzymatically.

14. The method of claim 13 wherein said conversion of glyoxylate to hydrogen peroxide is performed in the presence of glyoxal oxidase.

15. The method of claim 13 wherein said conversion of glyoxylate to hydrogen peroxide is performed in the presence of glycolate oxidase.

16. A method for detecting the presence of compounds having a glycine residue, in free acid form and attached to a carbonyl group, said method comprising the steps of:
(A) contacting a sample suspected of containing said glycine compounds with either (i) peptidylglycine alpha-amidating monooxygenase, (ii) a combination of peptidylglycine alpha-hydroxylating monooxygenase and peptidyl alpha-hydroxyglycine alpha-amidating lyase or (iii) a combination of peptidylglycine alpha-hydroxylating monooxygenase and a Lewis base; to form an amidated compound and glyoxylate;
(B) assaying for the presence of glyoxylate.

17. The method of claim 16 wherein said assay for glyoxylate comprises the steps of:
(A) contacting said test sample with one or more agents that convert glyoxylate, directly or indirectly, to hydrogen peroxide as one reaction product; and
(B) assaying for the presence of hydrogen peroxide.

18. The method of claim 17 wherein hydrogen peroxide is detected by a fluorescent assay.

19. The method of claim 18 wherein said fluorescent assay comprises reacting a fluorophore with a sample suspected of containing hydrogen peroxide to form a peroxide-dependent fluorescent product when hydrogen peroxide is present.

20. The method of claim 19 wherein said flurophore is amplex red, and said fluorescent product is resorufin.

21. The method of claim 17 wherein said conversion of glyoxylate to hydrogen peroxide is performed enzymatically.

22. The method of claim 21 wherein said conversion of glyoxylate to hydrogen peroxide is performed in the presence of glyoxal oxidase.

23. The method of claim 21 wherein said conversion of glyoxylate to hydrogen peroxide is performed in the presence of glycolate oxidase.

24. The method of claim 17 wherein hydrogen peroxide is detected by a luminescent assay.

25. The method of claim 24 wherein said luminescent assay comprises reacting luminol with a sample suspected of containing hydrogen peroxide.

26. The method of claim 25 wherein luminol is reacted at basic pH and in the presence on oxidative catalyst.

27. The method of claim 26 wherein oxidative catalyst is horseradish peroxidase.

28. The method of claim 25 wherein luminol is reacted at a pH no lower than 10.5.

29. The method of claim 24 wherein said conversion of glyoxylate to hydrogen peroxide is performed enzymatically.

30. The method of claim 29 wherein said conversion of glyoxylate to hydrogen peroxide is performed in the presence of glyoxal oxidase.

31. The method of claim 29 wherein said conversion of glyoxylate to hydrogen peroxide is performed in the presence of glycolate oxidase.

32. A method of identifying amidated hormones or amidated fatty acids comprising the steps of:
(A) incubating cells suspected of producing said hormones or fatty acids;
(B) fractionating compounds produced by said cells into a series of fractions for further analysis;
(C) contacting a fraction from step (B) with either (i) peptidylglycine alpha-amidating monooxygenase, (ii) a combination of peptidylglycine alpha-hydroxylating monooxygenase and peptidyl alpha-hydroxyglycine alpha-amidating lyase or (iii) a combination of peptidylglycine alpha-hydroxylating monooxygenase and a Lewis base; to form an amidated compound and glyoxylate when said fraction includes a compound having a glycine residue, in free acid form and attached to a carbonyl group; and
(D) Assaying the product of step (C) for the presence of glyoxylate.

33. The method of claim 32 wherein said assay for glyoxylate comprises the steps of:
(a) contacting said test sample with one or more agents that convert glyoxylate, directly or indirectly, to hydrogen peroxide as one reaction product; and
(b) assaying for the presence of hydrogen peroxide.

34. The method of claim 32 wherein said incubation occurs in the presence of an inhibitor of peptidylglycine alpha-amidating monooxygenase.

35. The method of claim 32 wherein a secretagogue is utilized to increase secretion into culture media of compounds produced by said cells.

36. The method of claim 32 wherein step (C) is performed in the absence of catalase.

37. The method of claim 36 wherein step (C) is performed in the presence of either a keto-acid or horseradish peroxidase.

38. The method of claim 32 wherein step (C) is performed in the absence of ascorbate.

39 The method of claim 38 wherein step (C) is performed in the presence of catechol.

40. The method of claim 32 wherein step (C) is performed in the presence of ascorbate, and ascorbate is deactivated or removed prior to step (D).

41. The method of claim 39 wherein said assay for glyoxalate is a fluorescent assay.

42. The method of claim 40 wherein said assay for glyoxalate is a luminescent assay.

43. The method of claim 32 wherein compounds in any fraction that, following step (D), tests positive for glyoxylate, are analyzed for characteristics of amidated peptide hormone precursors, said characteristics being selected from the group consisting of (1) having a C-terminal glycine in its amino acid sequence and (2) having, in its corresponding nucleotide sequence, a signal region in reading frame with a peptide coding region that either terminates with C-terminal glycine or has a glycine followed by single or multiple basic amino acids immediately downstream that serve as post-translational cleavage sites.

44. The method of claim 32 wherein, for any first sample of a fraction that, following step (D), tests positive for glyoxylate, a second sample of that fraction is subjected to steps (A) and (B), with the product of step (B) being assayed for the presence of glyoxylate to determine if the amount of glyoxalate detected for said second sample is desirably lower than the amount of glyoxalate detected for said first sample.
